(19) United States
(12) Patent Application Publication
(10) Pub. No.: US 2007/0003581 A1
(43) Pub. Date: Jan. 4, 2007

(54) Microemulsion for cosmetic or pharmaceutical use containing an active ingredient

(76) Inventor: Monna Marie Manning, Columbus, OH (US)

Correspondence Address: FROST BROWN TODD, LLC, 2200 PNC CENTER, 201 E. FIFTH STREET, CINCINNATI, OH, 45202, US

(21) Appl. No.: 11/515,308

(22) Filed: Sep. 1, 2006

Related U.S. Application Data
(63) Continuation of application No. 10/378,021, filed on Mar. 4, 2003.

(51) Int. Cl. A61K 9/00

(52) U.S. Cl. 424/400

(57) ABSTRACT

The invention relates to a transparent microemulsion comprised of a surfactant or emulsifier based on polyethylene glycol, a first cosurfactant (or coemulsifier) based on polyglycerol, a second cosurfactant (or coemulsifier) based on monoesters, an aqueous or hydrophilic phase, and may or may not include an active for delivery that can include vitamins, sunscreens, antiaging agents, or pharmaceutical actives. The said microemulsion is useful in personal care and pharmaceutical products.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

The present invention relates to clear microemulsion blends having average particle sizes that are less than the wavelength of light. The blends can be diluted to various levels to make them less costly or can be made more concentrated depending upon the actives they contain. The microemulsion blends of the present invention are thermodynamically stable and are useful as personal care or pharmaceutical delivery vehicles.

The personal care market currently offers products to the consumer in many forms. One group of products can be characterized as emulsions. Emulsions are dispersions containing at least two immiscible phases, one of which is dispersed in the form of small droplets throughout the other. Oil-in-water and water-in-oil emulsions are well known vehicles that are directed at improving the health or appearance of the skin. In particular, cosmetic products sold in emulsion form include lotions, creams, serums, gels, tonics, and pastes. These products are often directed at delaying or minimizing skin wrinkling and other histological changes often associated with skin aging or the results of environmental damage. Personal care products that treat such problems as increased pore size; skin roughness or redness or fine lines are generally opaque to semi-transparent but can include transparent forms. Transparent lotions are often preferred because they provide moistness or softness to the skin without a greasy or sticky after-feel. However, as the actives to be delivered to the skin to ameliorate certain skin conditions are increased, a loss of transparency can occur as well as an increased sticky or greasy feel. It is also well known in the art that surfactants and cosurfactants, useful in emulsifying actives to be delivered to the skin, may cause irritation at the increased levels required to make such a product thermodynamically stable.

European patent 334,777 A1 discloses a microemulsion suitable for use in a cosmetic or pharmaceutical application. Unfortunately, a lipid phase, such as mineral oil or vegetable oil, is an essential component of the invention. There are many instances in the market place where such a lipid is unwanted. For example, in the preparation of a skin care formulation for the treatment of acne, mineral oil or any other oil would not be desired. Even though a microemulsion would be beneficial in that the particle size for any active drug would be small, having to also include a comodegenic substance such as mineral oil into the product would not be commercially viable.

Accordingly, it is the object of the present invention to provide an improved microemulsion formulation which is advantageously transparent and stable in character and which can be used at dilute levels in economical formulations but can also be formulated to deliver effective levels of actives to the skin without causing a greasy or sticky feeling to the consumer for use in personal care or pharmaceutical products.

U.S. Pat. No. 5,252,555 gives many examples of microemulsions similar to those disclosed in EP 334,777 A1, previously discussed. Example 3 of U.S. Pat. No. 5,252,555 teaches the preparation of a microemulsion with the absence of a lipid phase. However, an anionic surfactant is an essential component, in particular, a phosphate that is essentially a strong detergent. We wish to exclude any strong anionic surfactants. Although, U.S. Pat. No. 5,252,555 teaches the preparation of a microemulsion without a lipid phase such as mineral oil or vegetable oil, the employment of an anionic surfactant is not advantageous in the marketplace and has little commercial value. An anionic surfactant is excluded from the present invention due to detergency, protein binding and association, as well as disruption of the skin barrier function. It is not difficult to imagine that an anionic surfactant might closely associate with an active ingredient and have the potential to render it ineffectual.

Still another object of this invention is to provide a microemulsion blend with improved skin mildness and low toxicity, that does not cause skin irritation even at higher use levels of surfactants and cosurfactants.

### BRIEF SUMMARY OF THE INVENTION

The present invention is a transparent microemulsion comprised of a blend of:
(a) An aqueous or hydrophilic phase,
(b) At least one surfactant or emulsifier based on polyethylene glycol,
(c) A first cosurfactant (or coemulsifier) based on polyglycerol,
(d) A second cosurfactant (or coemulsifier) based on monoesters,
(e) And may or may not include an active for delivery to the skin, orally, rectally, injected or through other mucous membranes that can include vitamins, sunscreens, anti-aging agents, pharmaceutical actives, etc.

In the description and claims, by surfactant, also called surface-active agents, one indicates a group of chemical substances that exhibit the ability to lower the surface tension of water or to reduce the interfacial tension between two immiscible substances.

### BRIEF DESCRIPTION OF THE SEVERAL VIEWS OF THE DRAWING

FIG. 1: Phase Diagram Depicting An Area of Microemulsion.

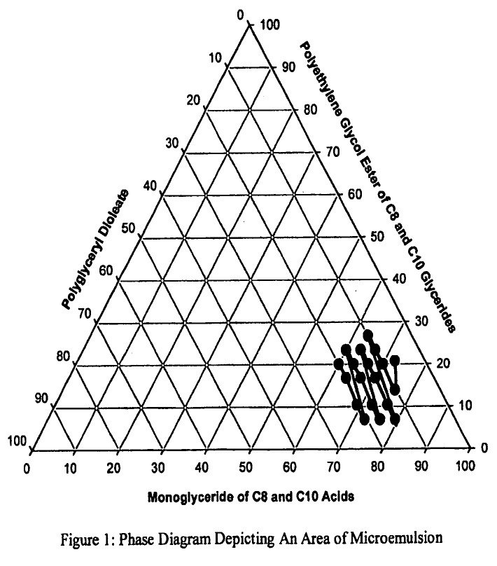

To determine the ratios of surfactant to cosurfactants when emulsifying a greater amount of cosmetic or pharmaceutical active into a transparent microemulsion, it is often helpful to make a phase diagram FIG. 1 shows the ratio of polyethylene glycol ester of C₈-C₁₀ glycerides surfactant to polyglyceryl dioleate cosurfactant to monoglyceride of C8 and C10 acids cosurfactant. The values of the three ingredients have been normalized to 100 percent. The diagram is based on emulsifying 5% by weight of Tocopheryl Acetate into a clear microemulsion and the addition of approximately 65% by weight of demineralized water.

### DETAILED DESCRIPTION OF THE INVENTION

It is well known that a microemulsion like that described above consists of a homogenous and virtually transparent micellar solution containing particles between approximately 5 to 500 (10⁻¹⁰) Angstroms in size.

It is also known that both the surfactant and two cosurfactants are used as emulsifying agents and, in some cases, solubilizing agents for actives delivery.

In European Patent EP 334,777 A1, it was established that a microemulsion for use in the cosmetics industry, comprised, as in the present instance, a hydrophilic phase (such as water, glycols, glycerol or mixtures of these compounds), a surfactant based on polyethylene glycol and a cosurfactant based on polyglycerol. However, it will be established in this document that the hydrophilic phase was necessarily combined with an oily phase of lipophilic nature, such as mineral, animal or synthetic oil.

In the context of the present invention it was found that such a lipid portion was nonessential, and that a stable microemulsion could be formed without a fatty phase of lipophilic nature.

In this context the definition of lipophilic will apply to a molecular group which is non-polar overall, and which has attraction or affininities toward molecules of an organic medium having a predominantly hydrophobic character, and in particular lipids, these lipids being defined as lipophilic substances which are insoluble in water and soluble in, for example, benzene and ethers, and which contain one or more fatty acids or fatty acid derivatives such as esters, alcohols and aldehydes.

Further, in publications EP 334, 777 A1 and EP 334,777, B1, it is also stated that if the “surfactants” are hydrophilic in character, their use will result only in micelle formation and that a surfactant used by itself only permits the formation of a micelle solution. Also, it is stated that cosurfactants are in character more hydrophobic and are necessary to cause the mutual solubilization of the aqueous and oil phases necessary to form a microemulsion.

It has been found that, in addition to a hydrophilic surfactant, that a system of two cosurfactants works best in forming a microemulsion, instead of just one. Further, one of the cosurfactants may be used to solubilize an active ingredient into the microemulsion formulation for delivery of the active agent to the skin, orally, rectally, injected or through the mucous membranes. Such an active may or may not be water-soluble, but is not necessary for the formation of the microemulsion.

In both publications EP 334,777 B1, EP 334,777 A1 and in U.S. Pat. No. 5,252,555, it is stated that, in regards to the surfactant or primary emulsifier used, it is most advantageously a mixed ester of glycerol and polyethylene glycol, present to the extent of at least approximately 10% by weight of the mixture. This weight percent being dictated in practice by the fact that, below this content, there is a tendency to obtain simply a traditional that is to say opaque, emulsion instead of a microemulsion. In the present invention, with the novel employment of two cosurfactants in addition to the primary surfactant, one is able to achieve a microemulsion with levels of the primary surfactant at levels as low as 8% by weight.

The microemulsion hereby obtained is particularly adapted to the pharmaceutical or cosmetic industry, is safe to use, well tolerated by the skin, non-toxic and has practically no detergent action.

Advantageously, in practice, the surfactant or primary emulsifier used is a mixed ester of glycerol and polyethylene glycol present to the extent of at least approximately 8% by weight of the mixture, this limit being dictated by the fact that, below this boundary there is a tendency to obtain an opaque emulsion instead of a microemulsion. The upper limit of use of the surfactant will essentially be dictated by the concentration of actives in the blend. The surfactant or primary emulsifier will advantageously have an average molecular weight of between 100 and 1000, preferably between 200 and 400, and even more preferably either 300 or 400, giving the surfactant a non-detergent character. These esters have a combined surfactant and skin feel function.

Advantageously, in practice, the first emulsion cosurfactant employed is an ester of polyglycerol and a fatty acid, which is liquid at room temperature. This cosurfactant can be employed in smaller concentrations of use than that of the abovementioned surfactant, preferably between approximately 0.1 and 75% by weight of the mixture.

More preferably, the cosurfactant will be employed at 0.3 to 50% by weight of the mixture. Most preferably, the cosurfactant will be employed at 0.5 to 20% by weight of the mixture.

The second emulsion cosurfactant will be advantageously, in practice, a monoglyceride of medium chain acids, preferably a mixture of C8 and C10 acids. The second cosurfactant can be used in smaller concentrations than the abovementioned surfactant, preferably between 0.1 and 75% by weight of the mixture. More preferably, the second cosurfactant will be employed at 0.3 to 50% by weight of the mixture. Most preferably, the second cosurfactant will be employed at 0.5 to 20% by weight of the mixture.

As regards to the actives that can be emulsified into the microemulsion, the list of potential ingredients can include any and all products that may ameliorate the condition or appearance of the skin or which may protect the skin from the damaging effects of the environment, or lessen the appearance of such damage. Also includes are substances which decrease the effects of aging on the appearance of the skin or which may reverse these effects in some way. Included in this category are sunscreens such as ethylhexyl salicylate, benzophenone-3, benzophenone-4, ethylhexyl methoxycinnamate, and homosalate, etc. Also included are vitamins and their derivatives such as tocopheryl acetate, retinol, etc. Further this would include botanical or naturally occurring extracts, oils, or fermentation products. It would also include hydroxyquinone, cholesterol, ceramides, sphingolipids, enzymes and other synthetically produced organic additives.

Actives in the case of this invention can also include any pharmaceutical agents that are required to be delivered through the skin, orally, rectally, injected or through the mucous membranes.

Humectants of the polyhydric alcohol type may be included in the aqueous phase of compositions according to the present invention. Amount of these humectants may range from 0.5 to about 10% by weight of the microemulsion. Illustrative of this category are propylene glycol, dipropylene glycol, sorbitol, glycerin and mixtures or derivatives thereof.

Preservatives are usually incorporated into cosmetic compositions of this invention at levels ranging from 0.01 to 3%, preferably from about 0.3 to about 2%. Particularly preferred preservatives are methylparaben, propylparaben, disodium edetate, imidazolidinyl urea and combinations thereof.

Minor adjunct ingredients may also be present in amounts from 0.01 to 2% by weight each. These can include antioxidants, antifoam agents, opacifiers and colorants, each in an effective amount to accomplish their respective function.

The manner by which the invention is achieved and the advantages that are derived are better understood with some actual examples which follow, given without implied limitation:

### EXAMPLE 1

| | |
|---|---|
| 8.0 | grams of a polyethylene glycol ester of C₈-C₁₀ glycerides |
| | with polyethylene glycol having a molecular weight of |
| | approximately 300 (sold under the tradename |
| | Acconon ® CC-6) |
| 1.0 | gram of a cosurfactant polyglyceryl dioleate (sold under |
| | the tradename Capmul ® MPGO) |
| 1.0 | gram of a cosurfactant monoglyceride of C8 and C10 acids |
| | (sold under the tradename Capmul ® MCM) |
| qs to 100 | grams of demineralized water |

At room temperature, the monoglyceride of C8 and C10 acids cosurfactant is blended until even with the polyglyceryl dioleate cosurfactant and then the polyethylene glycol ester of C8-C10 glycerides is combined and the blend agitated until even. Demineralized water is then added to the blend to make the microemulsion. Stirring was continued until a transparent microemulsion was formed. Preservatives and/or perfumes may be added as required but were not utilized in this example.

This formulation can be used alone as an economical spray lotion or can be used as a vehicle for actives delivery in the pharmaceutical or cosmetics industry.

### EXAMPLE 2

The following polar oils were each combined with the formulation in Example #1 and each formed a clear, stable microemulsion at room temperature:

| | |
|---|---|
| 1.0 | gram Hamamelis Virginiana (Witch Hazel) Extract |
| 1.0 | gram Eucalyptus Oil |
| 1.0 | gram Octyl Salicylate |
| 1.0 | gram Tocopheryl Acetate |

At room temperature, each active listed above is solubilized in the monoglyceride of C8 and C10 acids cosurfactant with stirring. The polyglyceryl dioleate cosurfactant is then added with agitation and again stirred until even. Next, the polyethylene glycol ester of C8 and C10 glycerides is combined with the blend and agitated until even. Demineralized water is then added to the blend to form the microemulsion. Stirring is continued until a transparent microemulsion is formed.

### EXAMPLE 3

| | |
|---|---|
| 19.0 | grams of a polyethylene glycol ester of C₈-C₁₀ glycerides |
| | with polyethylene glycol of a molecular weight of |
| | approximately 400 (sold under the tradename |
| | Acconon ® MC-8) |
| 4.5 | grams of a cosurfactant polyglyceryl dioleate (sold |
| | under the tradename Capmul ® MPGO) |
| 4.5 | grams of a cosurfactant monoglyceride of C8 and C10 acids |
| | (sold under the tradename Capmul ® MCM) |
| qs to 100 | grams of demineralized water |

At room temperature, the monoglyceride of C8 and C10 acids cosurfactant is blended until even with the polyglyceryl dioleate cosurfactant and then the polyethylene glycol ester of C8 and C10 glycerides is combined to the blend and agitated until even. Demineralized water is then added to the blend to make the microemulsion. Stirring was continued until a transparent microemulsion was formed. Preservatives and/or perfumes may be added as required but were not utilized in this example.

This formulation may be used alone as an economical spray lotion or can be used as a vehicle for actives delivery in the pharmaceutical or cosmetics industry.

Based on FIG. 1 Phase Diagram Depicting An Area of Microemulsion, the following microemulsion was prepared:

### EXAMPLE 4

| | |
|---|---|
| 21.0 | grams of a polyethylene glycol ester of C₈-C₁₀ glycerides |
| | with polyethylene glycol having a molecular weight of |
| | approximately 300 (sold under the tradename |
| | Acconon ® CC-6) |
| 4.0 | grams of a cosurfactant polyglyceryl dioleate (sold |
| | under the tradename Capmul ® MPGO) |
| 5.0 | grams of a cosurfactant monoglyceride of C8 and C10 acids |
| | (sold under the tradename Capmul ® MCM) |
| 5.0 | grams of Tocopheryl Acetate |
| qs to 100 | grams of demineralized water |

In the above example, at room temperature, tocopheryl acetate is first solubilized in the monoglyceride of C8 to C10 acids cosurfactant with stirring. The polyglyceryl dioleate cosurfactant is then added with agitation and again stirred until even. Next, the polyethylene glycol ester of C8 to C10 glycerides is combined with the blend and agitated until even. Demineralized water is then added to the blend to form the microemulsion. Stirring is continued until a transparent microemulsion is formed. Preservatives and perfumes may be added as required but were not utilized in this example.

In addition to a traditional microemulsion, the emulsions based upon these claims can be used in mousses, pressurized forms, aerosols, gel capsules, ampoules, etc.

These microemulsions can be successfully used in all cosmetic applications or external cutaneous pharmaceuticals (topicals), internal (oral rectal, injectable), or as excipients with good physiological compatibility. They also have excellent stability at the temperatures in which such products are normally used (that is to say in the range of −30° C. to +80° C., approximately).

What is claimed in this invention is:

1. A microemulsion for pharmaceutical or cosmetic use, comprising a mixture of:
at least one water-soluble phase, qs to 100.
at least one surfactant or emulsifier based on polyethylene glycol, and represents at least 8% to 50% by weight of the mixture;
at least a cosurfactant based on polyglycerol and fatty acids, which may be, in the mixture, in a proportion lower than that of the surface-active agent, and represents at least 0.5% to 20% by weight of the mixture;
at least a second cosurfactant based on monoesters of medium chain acids, which may be, in the mixture, in a proportion lower than that of the surface-active agent, and represents at least 0.5% to 20% by weight of the mixture.

2. The microemulsion set forth in claim 1, wherein the hydrophilic compound is selected from the group consisting of water, glycols, alcohols, polyols, humectants, extracts and other skin active agents, alone or in mixture.

3. The microemulsion set forth in claim 1, wherein the polyethyleneglycol of the surface-active agent has a molecular weight close to 300.

4. The microemulsion set forth in claim 1 wherein the polyethyleneglycol of the surface-active agent has a molecular weight close to 400.

5. The microemulsion set forth in claim 1-4 wherein the cosurfactant based on polyglycerol has a molecular weight of between 100 and 1000.

6. The microemulsion of claim 5, wherein the fatty acid is selected from a group comprising oleic acid and isostearic acid or other fatty acids which are liquid at room temperature.

7. The microemulsion of one of the claims 1 to 6 wherein the mixture also comprises cosmetic or pharmaceutical active ingredients, either alone or in solution in a solvent, which is either oil or water-soluble.

8. The microemulsion of claim 7, wherein the cosmetic or pharmaceutical active ingredients may be delivered cutaneously, subcutaneously, orally, rectally, injected, or through any mucous membrane.

9. The microemulsion of claims 1-6, wherein the mixture also contains vitamins or vitamin derivatives as the active ingredient.

10. The microemulsion of claim 1, wherein the mixture also comprises compounds selected from a group constituted by preservation agents, perfumes, biological or plant derivatives, and cosmetic additives.

11. The microemulsion of claim 1, wherein the mixture also comprises other ingredients known to the art to make the final formulation more aesthetically acceptable, as with thickening or viscosifying agents, opacifiers, colorant additives, etc.
